(19) United States
(12) Patent Application Publication
(10) Pub. No.: US 2018/0022983 A1
(43) Pub. Date: Jan. 25, 2018

(54) FLAVONOID AND PLANT PHENOLIC ACID ADDITIVES FOR DOWNHOLE FLUIDS

(71) Applicant: Charles D. Armstrong, Tomball, TX (US)

(72) Inventor: Charles D. Armstrong, Tomball, TX (US)

(73) Assignee: Baker Hughes, a GE company, LLC, Houston, TX (US)

(21) Appl. No.: 15/646,598

(22) Filed: Jul. 11, 2017

Related U.S. Application Data
(60) Provisional application No. 62/364,158, filed on Jul. 19, 2016.

(51) Int. Cl. C09K 8/52; C07H 15/26; C09K 8/62; C09K 8/035; C09K 8/58

(52) U.S. Cl. CPC C09K 8/52; C09K 8/035; C09K 8/58; C09K 8/62; C07H 15/26; C09K 2208/22

(57) ABSTRACT

A downhole fluid includes an additive composition, wherein the additive composition includes a flavonoid or a plant phenolic acid. A method includes circulating a downhole fluid composition into a subterranean reservoir wellbore, wherein the downhole fluid composition includes an additive composition, wherein the additive composition includes a flavonoid or a plant phenolic acid

### CROSS REFERENCE TO RELATED APPLICATIONS

This application claims the benefit of an earlier filing date from U.S. Provisional Application Ser. No. 62/364,158 Jul. 19, 2016, the entire disclosure of which is incorporated herein by reference.

### BACKGROUND

Various fluids are used during downhole operations, and fluid additives are used to prevent freezing of the downhole fluids as well as to reduce the solids that can plug or block fluid pathways, reducing flow. For example, a number of hydrocarbons, especially lower-boiling light hydrocarbons in formation fluids or natural gas are known to form hydrates in conjunction with water present under a variety of conditions particularly at the combination of lower temperature and higher pressure. The hydrates usually exist in solid forms that are essentially insoluble in the fluid itself. The solid hydrates may cause issues for production, handling, and transport of these fluids. For example, hydrate solids (or crystals) may cause plugging and/or blockage of pipelines, transfer lines, other conduits, valves, safety devices, and/or other equipment, which may result in shutdown, loss of production, risk of explosion, or unintended release of hydrocarbons into the environment either on-land or off-shore.

Low Dosage Hydrate Inhibitors (LDHIs) are used to overcome or control the hydrocarbon hydrate problems. One subset of LDHIs are the kinetic hydrate inhibitors (KHIs). KHIs inhibit, retard and/or prevent initial hydrocarbon hydrate crystal nucleation; and/or crystal growth. Exemplary KHIs include onium compounds with at least four carbon substituents used to inhibit the plugging of conduits by gas hydrates. Additives such as polymers with lactam rings have also been employed to control clathrate hydrates in fluid systems.

Another problem in oil drilling and exploration involves the formation of scale. Scale is a deposit or coating formed on the surface of metal, rock or other materials. Scale formation may be caused by a precipitation from a chemical reaction with the surface, precipitation caused by chemical reactions, a change in pressure or temperature, or a change in the composition of a solution. Typical scales are calcium carbonate, calcium sulfate, barium sulfate, strontium sulfate, iron sulfide, iron oxides, iron carbonate, various silicates and phosphates and oxides, or any of a number of compounds insoluble or slightly soluble in water.

Hydrate and/or scale formation are deleterious in many downhole fluids, such as drilling fluids, completion fluids, servicing fluids, fracturing fluids, production fluids, injection fluids, and combinations thereof.

While the currently available additives and KHIs are suitable for their intended purpose, it would be desirable to provide additives and KHIs that are natural products, such as molecules naturally found in plants.

### BRIEF SUMMARY

In one aspect, a downhole fluid comprises an additive composition, wherein the additive composition comprises a flavonoid or a plant phenolic acid.

In another aspect, a method comprises circulating a downhole fluid composition into a subterranean reservoir wellbore, wherein the downhole fluid composition comprises an additive composition, wherein the additive composition comprises a flavonoid or a plant phenolic acid.

### DETAILED DESCRIPTION

A detailed description of one or more embodiments of the disclosed compositions and methods are presented herein by way of exemplification and not limitation.

It has been discovered that flavonoids and/or phenolic acids, particularly those naturally found in plants, may be added to downhole fluids, to depress the freezing point of the fluids. In addition, the flavonoids and phenolic acids may decrease or minimize an amount of at least one substance within the fluid composition, such as but not limited to, hydrate formation, scale, and combinations thereof. In addition to being biodegradable, the flavonoids and phenolic acids may be less toxic to the environment and may be made from renewable resources. The use of flavonoids and phenolic acids in a base fluid may provide a renewable alternative to conventional non-biodegradable additives that are used in downhole fluids to depress freezing point, decrease hydrate formation, scale, and combinations thereof.

Without being held to theory, it is believed that the flavonol glycosides, particularly the 7-position glucose or galactose substituted quercetins and kaempferols, will be particularly useful as pour point depressants and kinetic hydrate inhibitors, for example. These molecules have been shown to reduce the freezing point of water by as much as 16° C., while not changing the melting point, which makes then favorable for use in downhole fluids.

In one aspect, a downhole fluid comprises an additive composition, wherein the additive composition comprises a flavonoid or a plant phenolic acid.

Flavonoids are a large group of secondary metabolites in plants. Flavonoids include anthoxanthins, flavanones, flavanolols, flavans, isoflavanoids, neoflavonoids, anthocyandins, and polymers thereof. Anthoxanthins include flavones (e.g., luteolin, apigenin and tangeritin) and flavonols (e.g., quercetin, kaempferol, myricetin, fisetin, galangin, isorhamnetin, pachypodol, rhamnazin, and the like). Flavanones include hesperetin, naringenin, eriodictyol and homoeriodictyol. Flavonolols include catechins and epicatechins. Flavans include flavan-3ols, flavan-4-ols and flavan-3,4-diols. Isoflavanoids include genistein, daidzein, glycitein. Neoflavonoids are similar to flavonoids but have a 4-phenylchromen backbone with no hydroxyl group substitution at position 2. Anthocyanidins include cyanidin, delphinidin, malvidin, pelargonidin, peonidin, and petunidin. Polymers include proanthocyanidins. A specific flavonoid is a flavonol glycoside.

Phenolic acids are also found in many plant species. Exemplary plant phenolic acids include cinnamic acid, o-coumaric acid, p-coumaric acid, m-coumaric acid, ferulic acid, sinapic acid, caffeic acid, benzoic acid, salicylic acid, p-hydroxybenzoic acid, vanillic acid, syringic acid, procatechechuic acid, gentisic acid, gallic acid, veratric acid, and the like.

In one aspect, the additive is a flavonol glycoside. In one aspect, the flavonol glycoside is naturally found in plants.

In one aspect, the flavonol glycoside has Formula 1,

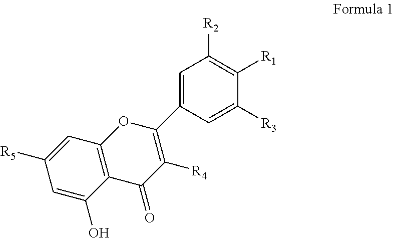

wherein R¹, R² and R³ are each independently H, OH or OCH₃; R⁴ and R⁵ are each H or OH, and one of R⁴ and R⁵ is OR⁶, wherein R⁶ is a sugar. Exemplary sugars are glucose, rhamnose, galactose, xylose and arabinose.

In the kaempferols, R¹ is OH and R² and R³ are H. In the quercetins, R¹ and R² are OH, and R³ is H. In the myrecetins, R¹, R² and R³ are OH. In an exemplary embodiment, the flavonol glycoside is a kaempferol, a quercetin, or a combination thereof. Exemplary flavonol glycosides include rutin, quercimetrin, cyaroside and hesperidin, and combinations thereof.

Downhole fluids are typically classified according to their base fluid. In water-based fluids, solid particles, such as weighting agents, are suspended in a continuous phase of water or brine. Oil can be emulsified in the water, which is the continuous phase. “Water-based-fluid” is used herein to include fluids having an aqueous continuous phase where the aqueous continuous phase can be all water or brine, an oil-in-water emulsion, or an oil-in-brine emulsion. Brine-based fluids, are water-based fluids, in which the aqueous component is brine.

Exemplary downhole fluids include a drilling fluid, a completion fluid, a production fluid, a servicing fluid, a fracturing fluid, an injection fluid, a refinery fluid, and combinations thereof.

There are a variety of functions and characteristics that are expected of completion fluids. The completion fluid may be placed in a well to facilitate final operations prior to initiation of production. Completion fluids are typically brines, such as chlorides, bromides, and/or formates, and other non-damaging fluids having suitable density and flow characteristics. Exemplary salts for forming the brines include, but are not necessarily limited to, sodium chloride, calcium chloride, zinc chloride, potassium chloride, potassium bromide, sodium bromide, calcium bromide, zinc bromide, sodium formate, potassium formate, ammonium formate, cesium formate, and mixtures thereof. It is preferred that the completion fluid is chemically compatible with the reservoir formation and formation fluid. Chemical additives, such as polymers and surfactants may be introduced to the brines used in base fluids for various reasons that include, but are not limited to, increasing viscosity, and increasing the density of the brine. Completion fluids typically do not contain suspended solids.

Production fluid is the fluid that flows from a formation to the surface of an oil well. These fluids may include oil, gas, water, as well as any contaminants (e.g., H₂5, asphaltenes, etc.). The consistency and composition of the production fluid may vary.

Servicing fluids, such as remediation fluids, stimulation fluids, workover fluids, and the like, have several functions and characteristics suitable for repairing a damaged well. Such fluids may be used for breaking emulsions that are already formed and for removing formation damage that may have occurred during the drilling, completion and/or production operations. The terms “remedial operations” and “remediate” include a lowering of the viscosity of gel damage and/or the partial or complete removal of damage from a subterranean formation. Similarly, the term “remediation fluid” includes a fluid that may be useful in remedial operations. A stimulation fluid may be a treatment fluid prepared to stimulate, restore, or enhance the productivity of a well, such as fracturing fluids and/or matrix stimulation fluids in one non-limiting example.

Hydraulic fracturing is a type of stimulation operation, which uses pump rate and hydraulic pressure to fracture or crack a subterranean formation in a process for improving the recovery of hydrocarbons from the formation. Once the crack or cracks are made, high permeability proppant relative to the formation permeability is pumped into the fracture to prop open the crack. When the applied pump rates and pressures are reduced or removed from the formation, the crack or fracture cannot close or heal completely because the high permeability proppant keeps the crack open. The propped crack or fracture provides a high permeability path connecting the producing wellbore to a larger formation area to enhance the production of hydrocarbons.

The development of suitable fracturing fluids is a complex art because the fluids must simultaneously meet a number of conditions. For example, they must be stable at high temperatures and/or high pump rates and shear rates that can cause the fluids to degrade and prematurely settle out the proppant before the fracturing operation is complete. Various fluids have been developed, but most commercially used fracturing fluids are aqueous based liquids that have either been gelled or foamed to better suspend the proppants within the fluid.

Injection fluids may be used in enhanced oil recovery (EOR) operations, which are sophisticated procedures that use viscous forces and/or interfacial forces to increase the hydrocarbon production, e.g. crude oil, from oil reservoirs. The FOR procedures may be initiated at any time after the primary productive life of an oil reservoir when the oil production begins to decline. The efficiency of FOR operations may depend on reservoir temperature, pressure, depth, net pay, permeability, residual oil and water saturations, porosity, fluid properties, such as oil API gravity and viscosity, and the like.

Refinery fluids are fluids that may be further processed or refined at a refinery. A non-limiting example of a refinery process may include reducing or preventing the formation of foulants. Non-limiting examples of foulants may be or include hydrates, asphaltenes, coke, coke precursors, naphthenates, inorganic solid particles (e.g. sulfates, oxides, scale, and the like), and combinations thereof. Non-limiting examples of refinery fluids include crude oil, production water, and combinations thereof.

EOR operations are considered a secondary or tertiary method of hydrocarbon recovery and may be used when the primary and/or secondary recovery operation has left behind a substantial quantity of hydrocarbons in the subterranean formation. Primary methods of oil recovery use the natural energy of the reservoir to produce oil or gas and do not require external fluids or heat as a driving energy FOR methods are used to inject materials into the reservoir that are not normally present in the reservoir.

Secondary FOR methods of oil recovery inject external fluids into the reservoir, such as water and/or gas, to re-pressurize the reservoir and increase the oil displacement. Tertiary FOR methods include the injection of special fluids, such as chemicals, miscible gases and/or thermal energy. The FOR operations follow the primary operations and target the interplay of capillary and viscous forces within the reservoir. For example, in EOR operations, the energy for producing the remaining hydrocarbons from the subterranean formation may be supplied by the injection of fluids into the formation under pressure through one or more injection wells penetrating the formation, whereby the injection fluids drive the hydrocarbons to one or more producing wells penetrating the formation. EOR operations are typically performed by injecting the fluid through the injection well into the subterranean reservoir to restore formation pressure, improve oil displacement or fluid flow in the reservoir, and the like.

Examples of EOR operations include water-based flooding and gas injection methods. Water-based flooding may also be termed ‘chemical flooding’ if chemicals are added to the water-based injection fluid. Water-based flooding may be or include, polymer flooding, ASP (alkali/surfactant/polymer) flooding, SP (surfactant/polymer) flooding, low salinity water and microbial FOR; gas injection includes immiscible and miscible gas methods, such as carbon dioxide flooding, and the like.

The flavonoid or plant phenolic acid may be in a powder form and/or a liquid form (e.g., in solution) when added to or included in the base fluid. The flavonoid or plant phenolic acid may be part of an additive composition, where the additive composition includes the flavonoid or plant phenolic acid, as well as other components to aid the flavonoid or plant phenolic acid in depressing the freezing point, minimizing hydrate formation, and minimizing scale formation of the fluid composition.

The flavonoid, plant phenolic acid or additive composition may be added or present in the base fluid composition in an amount of about 0.01 vol % to about 10 vol % of the total base fluid, about 0.1 vol % to about 5 vol %, or about 0.25 vol % to about 3 vol %. Alternatively, the flavonoid or plant phenolic acid may be present within the additive composition in an amount of about 1 ppm to about 10 ppt as compared to the total amount of base fluid. The flavonoid or plant phenolic acid may depress the freezing point of the fluid composition by at least 0.5° C., alternatively from about 1° C. to about 30° C., alternatively from about 5° C. to about 20° C., or from about 10° C. to about 15° C.

The additive composition may also include a winterizing agent, a kinetic hydrate Inhibitor, a pour point depressant, an anti-agglomerant, common fracturing fluid additives, and combinations thereof. The winterizing agent may be or include, but is not limited to, alcohols, glycols, diols, or glycol ethers including those that generally contain a hydroxyl group or multiple hydroxyl groups. Such alcohols, glycols, diols, or glycol ethers may be selected from, in non-limiting embodiments, methanol, ethanol, propanol, isopropanol, butanol, ethylene glycol, propylene glycol, butylene glycol, hexylene glycol, diethylene glycol, dipropylene glycol, triethylene glycol, tripropylene glycol, propylene glycol methyl ether, dipropylene glycol methyl ether, tripropylene glycol methyl ether, propylene glycol propyl ether, dipropylene glycol propyl ether, tripropylene glycol propyl ether, propylene glycol butyl ether, dipropylene glycol butyl ether, tripropylene glycol butyl ether, propylene glycol phenyl ether, dipropylene glycol dimethyl ether, diethylene glycol ethyl ether, diethylene glycol methyl ether, diethylene glycol butyl ether, diethylene glycol hexyl ether, ethylene glycol propyl ether, ethylene glycol butyl ether, ethylene glycol hexyl ether, triethylene glycol methyl ether, triethylene glycol ethyl ether, triethylene glycol butyl ether, ethylene glycol phenyl ether, ethylene glycol ethyl ether, polyethylene glycol, monosaccharide, polypropylene glycol, dimethyl sulfoxide, glycerol, glycerine, copolymers thereof, and combinations thereof.

Once the flavonoid or plant phenolic acid (or an additive composition that includes the flavonol glycoside) is added to the base fluid, the resulting downhole fluid composition may be used in a downhole operation. In a non-limiting embodiment, the base fluid is an aqueous fluid, a non-aqueous fluid, or a combination thereof. In another non-limiting embodiment, the base fluid is contained in an oil pipeline, gas pipeline, a refinery (e.g., separation vessels, dehydration units, gas lines, and pipelines), or a combination thereof.

In a non-limiting embodiment, the downhole fluid composition is circulated into a subterranean reservoir wellbore, and the downhole fluid composition may have or include an effective amount of a flavonol glycoside to depress the freezing point of the downhole fluid composition relative to the melting point. The method may further include decreasing the amount of at least one substance within the fluid composition, such as but not limited to, hydrate formation, scale, and combinations thereof. Parameters that may be used to assess the effectiveness of hydrate inhibitors may include measurement of hydrate formation kinetics (rate of gas uptake), driving force measurements (e.g., the degree of sub-cooling required to initiate hydrate formation), and induction time. Methods for measuring these parameters may be used to assess the ability of the flavonol glycoside to reduce or inhibit hydrate formation. “Effective amount” is defined herein to mean an amount of flavonol glycoside that may depress the freezing temperature of the fluid composition; alternatively, “effective amount” is defined herein to mean an amount of the flavonol glycoside that may decrease the amount of scale, hydrates, or a combination thereof.

Determination of the induction time for hydrate formation in the presence or absence of flavonoid or plant phenolic acid may be used as an assay for the inhibition activity of the flavonol glycoside. Induction time may be the time required for the onset point of the crystallization. In this assay, samples may be kept at constant temperature with constant stirring and a sudden rise of temperature signals the onset point of crystallization of hydrates. The effect of a number of parameters including concentration of the flavonoid, plant phenolic acid, temperature, gas, and salt are determined and data subject to statistical analysis. Freezing points may be obtained with differential scanning calorimetry (DSC) as a measure of inhibition activity and for comparison with known kinetic inhibitors.

The growth rate of hydrates in the presence of the flavonoid or plant phenolic acid may also be determined and compared to activities of other flavonoids or plant phenolic acids and/or known kinetic inhibitors. Experiments may be performed by capturing images of the hydrate crystals growing in solution, using a specially built crystal-growth-observation apparatus. Effects of concentration of the flavonoid, plant phenolic acid, temperature, gas, and salt on the growth rate may also be examined.

The formation of scale in the presence of the flavonoid or plant phenolic acid may also be determined and compared to activities of other flavonoids, plant phenolic acids and/or known kinetic inhibitors. Experiments may be performed by capturing images of scale forming in solution. Effects of concentration of the flavonoid, plant phenolic acid, temperature, gas, and salt on the production of scale may also be examined.

Set forth below are some embodiments of the foregoing disclosure:

### Embodiment 1

A downhole fluid, comprising an additive composition, wherein the additive composition comprises a flavonoid or a plant phenolic acid.

### Embodiment 2

The downhole fluid of any prior embodiment, wherein the flavonoid is a flavonol glycoside naturally found in plants.

### Embodiment 3

The downhole fluid any prior embodiment, wherein the flavonol glycoside has Formula I

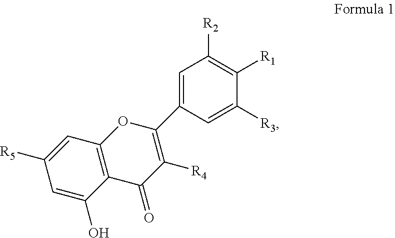

wherein R¹, R² and R³ are each independently H, OH or OCH₃; R⁴ and R⁵ are each H or OH, and one of R⁴ and R⁵ is OR⁶, wherein R⁶ is a sugar.

### Embodiment 4

The downhole fluid any prior embodiment, wherein the sugar is glucose, rhamnose, galactose, xylose and arabinose.

### Embodiment 5

The downhole fluid any prior embodiment, wherein the flavonol glycoside is a kaempferol and R¹ is OH and R² and R³ are H; or the flavonol glycoside is a quercetin and R¹ and R² are OH, and R³ is H.

### Embodiment 6

The downhole fluid any prior embodiment, wherein the flavonol glycoside is rutin, quercimetrin, cyaroside, hesperidin, myricetin, or a combinations thereof.

### Embodiment 7

The downhole fluid any prior embodiment, wherein the flavonoid or plant phenolic acid is added to a base fluid, and wherein the base fluid is a drilling fluid, a completion fluid, a production fluid, a servicing fluid, an injection fluid, a refinery fluid, or a combination thereof.

### Embodiment 8

The downhole fluid any prior embodiment, wherein the flavonoid or plant phenolic acid is in an amount of about 1 ppm to about 10 ppt as compared to the total base fluid.

### Embodiment 9

The downhole fluid any prior embodiment, further comprising an additional additive, wherein the additional additive is a winterizing agent, a kinetic hydrate inhibitor, a pour point depressant, an anti-agglomerant, a fracturing fluid additive, or a combination thereof.

### Embodiment 10

The downhole fluid of any prior embodiment, wherein the flavonoid is a flavone, a flavonol, a flavanone, a flavanolol, a flavan, an isoflavanoid, a neoflavonoid, an anthocyandin, a polymer, or a combination thereof.

### Embodiment 11

A method comprising, circulating a downhole fluid composition into a subterranean reservoir wellbore, wherein the downhole fluid composition comprises an additive composition, wherein the additive composition comprises a flavonoid or a plant phenolic acid.

### Embodiment 12

The method of any prior embodiment, wherein the flavonoid is a flavonol glycoside naturally found in plants.

### Embodiment 13

The method of any prior embodiment, wherein the flavonol glycoside has Formula I

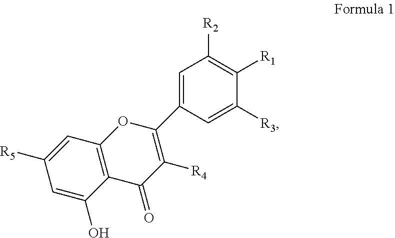

wherein R¹, R² and R³ are each independently H, OH or OCH₃; R⁴ and R⁵ are each H or OH, and one of R⁴ and R⁵ is OR⁶, wherein R⁶ is a sugar.

### Embodiment 14

The method of any prior embodiment, wherein the sugar is glucose, rhamnose, galactose, xylose and arabinose.

### Embodiment 15

The method of any prior embodiment, wherein the flavonol glycoside is a kaempferol and R¹ is OH and R² and R³ are H; or the flavonol glycoside is a quercetin and R¹ and R² are OH, and R³ is H.

### Embodiment 16

The method of any prior embodiment, wherein the flavonol glycoside is rutin, quercimetrin, cyaroside and hesperidin, or a combination thereof.

### Embodiment 17

The method of any prior embodiment, wherein the flavonoid or plant phenolic acid is added to a base fluid, and wherein the base fluid is a drilling fluid, a completion fluid, a production fluid, a servicing fluid, an injection fluid, a refinery fluid, or a combination thereof.

### Embodiment 18

The method of any prior embodiment, wherein the flavonoid or plant phenolic acid is in an amount of about 1 ppm to about 10 ppt as compared to the total base fluid.

### Embodiment 19

The method of any prior embodiment, further comprising an additional additive, wherein the additional additive is a winterizing agent, a kinetic hydrate inhibitor, a pour point depressant, an anti-agglomerant, a fracturing fluid additive, or a combination thereof.

### Embodiment 20

The method of any prior embodiment, wherein the flavonoid is a flavone, a flavonol, a flavanone, a flavanolol, a flavan, an isoflavanoid, a neoflavonoid, an anthocyandin, a polymer, or a combination thereof.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

The teachings of the present disclosure may be used in a variety of well operations. These operations may involve using one or more treatment agents to treat a formation, the fluids resident in a formation, a wellbore, and/or equipment in the wellbore, such as production tubing. The treatment agents may be in the form of liquids, gases, solids, semi-solids, and mixtures thereof. Illustrative treatment agents include, but are not limited to, fracturing fluids, acids, steam, water, brine, anti-corrosion agents, cement, permeability modifiers, drilling muds, emulsifiers, demulsifiers, tracers, flow improvers etc. Illustrative well operations include, but are not limited to, hydraulic fracturing, stimulation, tracer injection, cleaning, acidizing, steam injection, water flooding, cementing, etc.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A downhole fluid, comprising an additive composition, wherein the additive composition comprises a flavonoid or a plant phenolic acid.

2. The downhole fluid of claim 1, wherein the flavonoid is a flavonol glycoside naturally found in plants.

3. The downhole fluid of claim 2, wherein the flavonol glycoside has Formula I 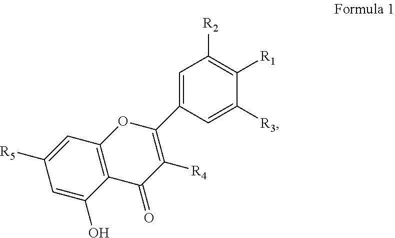
wherein R¹, R² and R³ are each independently H, OH or OCH₃; R⁴ and R⁵ are each H or OH, and one of R⁴ and R⁵ is OR⁶, wherein R⁶ is a sugar.

4. The downhole fluid of claim 3, wherein the sugar is glucose, rhamnose, galactose, xylose and arabinose.

5. The downhole fluid of claim 3, wherein the flavonol glycoside is a kaempferol and R¹ is OH and R² and R³ are H; or the flavonol glycoside is a quercetin and R¹ and R² are OH, and R³ is H.

6. The downhole fluid of claim 3, wherein the flavonol glycoside is rutin, quercimetrin, cyaroside, hesperidin, myricetin, or a combination thereof.

7. The downhole fluid of claim 1, wherein the flavonoid or plant phenolic acid is added to a base fluid, and wherein the base fluid is a drilling fluid, a completion fluid, a production fluid, a servicing fluid, an injection fluid, a refinery fluid, or a combination thereof.

8. The downhole fluid of claim 7, wherein the flavonoid or plant phenolic acid is in an amount of about 1 ppm to about 10 ppt as compared to the total base fluid.

9. The downhole fluid of claim 1, further comprising an additional additive, wherein the additional additive is a winterizing agent, a kinetic hydrate inhibitor, a pour point depressant, an anti-agglomerant, a fracturing fluid additive, or a combination thereof.

10. The downhole fluid of claim 1, wherein the flavonoid is a flavone, a flavonol, a flavanone, a flavanolol, a flavan, an isoflavanoid, a neoflavonoid, an anthocyandin, a polymer, or a combination thereof.

11. A method comprising, circulating a downhole fluid composition into a subterranean reservoir wellbore, wherein the downhole fluid composition comprises an additive composition, wherein the additive composition comprises a flavonoid or a plant phenolic acid.

12. The method of claim 11, wherein the flavonoid is a flavonol glycoside naturally found in plants.

13. The method of claim 12, wherein the flavonol glycoside has Formula I 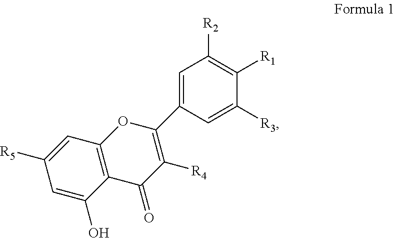
wherein R¹, R² and R³ are each independently H, OH or OCH₃; R⁴ and R⁵ are each H or OH, and one of R⁴ and R⁵ is OR⁶, wherein R⁶ is a sugar.

14. The method of claim 13, wherein the sugar is glucose, rhamnose, galactose, xylose and arabinose.

15. The method of claim 13, wherein the flavonol glycoside is a kaempferol and R¹ is OH and R² and R³ are H; or the flavonol glycoside is a quercetin and R¹ and R² are OH, and R³ is H.

16. The method of claim 13, wherein the flavonol glycoside is rutin, quercimetrin, cyaroside and hesperidin, or a combinations thereof.

17. The method of claim 11, wherein the flavonoid or plant phenolic acid is added to a base fluid, and wherein the base fluid is a drilling fluid, a completion fluid, a production fluid, a servicing fluid, an injection fluid, a refinery fluid, or a combination thereof.

18. The method of claim 17, wherein the flavonoid or plant phenolic acid is in an amount of about 1 ppm to about 10 ppt as compared to the total base fluid.

19. The method of claim 11, further comprising an additional additive, wherein the additional additive is a winterizing agent, a kinetic hydrate inhibitor, a pour point depressant, an anti-agglomerant, a fracturing fluid additive, or a combination thereof.

20. The method of claim 11, wherein the flavonoid is a flavone, a flavonol, a flavanone, a flavanolol, a flavan, an isoflavanoid, a neoflavonoid, an anthocyandin, a polymer, or a combination thereof.
